(19) United States
(12) Patent Application Publication
(10) Pub. No.: US 2019/0000754 A1
(43) Pub. Date: Jan. 3, 2019

(54) THERAPEUTIC COMPOSITIONS FOR THE TREATMENT OF DRY EYE AND RELATED OCULAR SURFACE DISEASES

(71) Applicant: Seinda Biomedical Corporation, Guangzhou (CN)

(72) Inventor: Jing-Feng HUANG, San Diego, CA (US)

(73) Assignee: Seinda Biomedical Corporation, Guangzhou (CN)

(21) Appl. No.: 15/766,361

(22) PCT Filed: Oct. 5, 2016
(86) PCT No.: PCT/US2016/055550

§ 371 Date: Apr. 5, 2018

Related U.S. Application Data
(60) Provisional application No. 62/237,494, filed on Oct. 5, 2015.

(51) Int. Cl. A61K 9/00; A61K 38/20; A61K 47/60; A61K 38/19; A61K 9/06; A61K 9/107; A61K 47/18; A61K 47/02; A61P 27/04

(52) U.S. Cl. CPC A61K 9/0048; A61K 38/20; A61K 47/60; A61K 38/191; A61P 27/04; A61K 9/06; A61K 9/107; A61K 47/186; A61K 47/02; A61K 38/2046

(57) ABSTRACT

Compositions, kits, and methods for treating for treating dry eyes in mammal, particularly humans, are described. Such compositions include a pharmaceutically or veterinarily acceptable ocular carrier and a therapeutically effective amount of one or more immune regulatory agents, for example, stimulators and activators of regulatory B or T cells, type 2 immunity-associated immune cells, including group 2 innate lymphoid cells, type 2 CD4+ T helper cells, and alternative activation of macrophages (AAMacs, M2).

### FIELD OF THE INVENTION

This invention relates generally to the field of ophthalmology.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

The ocular surface is continuously exposed to environmental agents such as allergens, pollutants, and microorganisms, which can provoke inflammation. The cornea and the underlying anterior chamber possess unique attributes that protect the cornea and the eye from immune-mediated inflammation and immune-mediated injury in the eye and create ocular immune tolerance, which is believed to be essential for maintaining normal vision and a healthy eye.

Dry eye disease (DED) is one of the most prevalent eye conditions, affecting millions of people in the United States alone and many millions more in other regions of the world. Ocular symptoms include dryness, ocular irritation and pain, and blurred vision, which can significantly affect quality of life and work-related activities. DED patients also report greater sensitivities and less tolerance to changes in their environments. Visual dysfunction includes difficulty in reading, driving, computer usage, watching TV, and other daily personal and work-related activities.

Dry eye symptoms have traditionally been managed with artificial tear eye drops, anti-inflammatory compounds (cyclosporine) and corticosteroids, topical antibiotics (erythromycin or bacitracin ointments) and oral tetracyclines (tetracycline, doxycycline, or minocycline), and eyelid hygiene, which are frequently ineffective, time consuming, frustrating, or variably effective treatments but with safety issues. Despite the high incidence rate of dry eye, topical cyclosporine-A (Restasis®) is the only approved treatment for DED in United States and it has high ocular irritation and low response rates. Thus, treating dry eye still remains a therapeutic challenge and as such, there is a significant need for developing new therapeutic modalities to treat DED.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” (or any other term of approximation) refers to approximately a +/−10% variation from the stated value. It is to be understood that such a variation is always included in any given value provided herein, whether or not it is specifically referred to.

The term “combination therapy” refers to a therapeutic regimen that involves the provision of at least two distinct therapies to achieve an indicated therapeutic effect. For example, a combination therapy may involve the administration of two or more chemically distinct active ingredients, for example, two or more immune regulatory agents. Alternatively, a combination therapy may involve the administration of an immune regulatory agent together with the delivery of another treatment, such as radiation therapy and/or surgery. Further, a combination therapy may involve administration of an immune regulatory agent together with one or more other biological agents (e.g., corticosteroid), anti-inflammatory agents, and/or another treatment such as radiation and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more immune regulatory agent species, alone or in conjunction with one or more other active ingredients are combined with, for example, radiation and/or surgery, the drug(s) may be delivered before or after surgery or radiation treatment.

The term “monotherapy” refers to a treatment regimen based on the delivery of one therapeutically effective compound, whether administered as a single dose or several doses over time.

A “patentable” process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

A “subject” is a member of any animal species, preferably a mammalian species, optionally a human. Other mammals include domestic and farm animals, and zoo, sports, or pet animals, such as dogs, horses, cats, sheep, pigs, cows, etc. Thus, the methods, compositions, reagents, and kits described herein are applicable to both human and veterinary dry eye disease. Preferred subjects are humans, and most preferably “patients,” which as used herein refers to living humans that are receiving medical care for a disease or condition. This includes persons with no defined illness who are being investigated for signs of pathology. The subject can be an apparently healthy individual, an individual suffering from a disease, or an individual being treated for dry eye disease. A “reference subject” or “reference subjects” is/are an individual or a population that serves as a reference against which to assess another individual or population with respect to one or more parameters.

A “therapeutically effective amount” (or “effective amount”) refers to an amount of an active ingredient (e.g., an immune regulatory agent according to the invention) sufficient to effect treatment when administered to a subject in need of such treatment. Accordingly, what constitutes a therapeutically effective amount of a composition may be readily determined by one of ordinary skill in the art. In the context of cancer therapy, a “therapeutically effective amount” is one that produces an objectively measured change in one or more parameters associated with dye eye disease, including an increase or decrease in the expression of one or more genes or biomarkers correlated therewith, disease stage or progression, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions described herein are used in methods of dry eye therapy. As used herein, the terms “therapy” and “therapeutic” encompasses the full spectrum of prevention and/or treatments for dry eye disease. A “therapeutic” agent may act in a manner that is prophylactic or preventive, including those that incorporate procedures designed to target individuals that can be identified as being at risk (e.g., via genetics, family history, etc.); or in a manner that is ameliorative or curative in nature; or may act to slow the rate or extent of the progression of at least one symptom of dry eye disease; or may act to minimize the time required, the occurrence or extent of any discomfort or pain, or physical limitations associated with recuperation from dry eye disease; or may be used as an adjuvant to other therapies and treatments.

The term “treatment” or “treating” means any treatment of dry eye disease or disorder, including preventing or protecting against the disease (that is, causing the clinical symptoms not to develop); inhibiting the disease (i.e., arresting, delaying or suppressing the development of clinical symptoms; and/or relieving the disease (i.e., causing the regression of clinical symptoms). As will be appreciated, it is not always possible to distinguish between “preventing” and “suppressing” dry disease because the ultimate inductive event or events may be unknown or latent. Those “in need of treatment” include those already having dry eye disease as well as those in which the disease is to be prevented. Accordingly, the term “prophylaxis” will be understood to constitute a type of “treatment” that encompasses both “preventing” and “suppressing”. The term “protection” thus includes “prophylaxis”.

### SUMMARY OF THE INVENTION

The present invention relates to ophthalmic formulations that contain one or more immune regulatory agents for use in the treatment and prevention of dry eye and related conditions of the ocular surface. The present invention also concerns methods for the therapeutic treatment and prevention of dry eye and related ocular surface conditions in a subject in need of such treatment, comprising administering the ophthalmic formulations of present invention directly to the eye or region of the eye of the subject. The subject is preferably a mammal in need of such treatment, e.g., a subject that has been diagnosed with dry eye disease of the ocular surface or a predisposition thereto. The mammal can be any mammal, e.g., a human, a primate, a dog, a cat, a horse, etc. In a preferred embodiment, the mammal is a human.

The present invention is based on the surprising, unexpected discovery that in the ocular surface of DED patients there is a significant loss of growth factors and cytokines that are associated with regulatory T cells (T_{reg}), regulatory B cells (B_{reg}), group 2 innate lymphoid cells (ILC2), type 2 CD4+ T helper cells (Th2), or, alternatively, activated macrophages (AAMacs or M2). These cytokines and growth factors include IL-10, IL-4, IL-5, IL-13, IL-3, GM-CSF, and lymphotoxin. Additionally, the significant loss or reduced levels of these growth factors and cytokines associated with T_{reg}, B_{reg} or type 2 immunity (ILC2, Th2, or AAMacs) in the tear film are further correlated with ocular surface tissue damage and decreased mucin production and goblet cell density in DED, and are accompanied by elevated levels of pathogenic inflammatory Th1 (Type 1 helper T cells), Th17 (Type 17 helper T cells), IL-8, and/or complement activation.

In vivo stimulation, expansion, and activation of T_{reg} and/or B_{reg} can result in suppression of inflammation, restoration of peripheral tolerance, and immune homeostasis. Stimulation and/or activation of innate ILC2s in vivo induces production of type 2 cytokines, including IL-4, IL5, IL-9, and IL-13, thus resulting in Th2 polarization, AAMacs induction and activation, resolution of inflammation, promotion of wound healing and tissue repair, enhancement of goblet cell differentiation, and mucin production. Stimulation and/or induction of AAMacs leads to clearance of apoptotic cells, suppression of inflammation, production of mucins and other extracellular matrix components, tissue repair, and restoration of tissue homeostasis.

The compositions of the invention (i.e., that contain one or more immune regulatory agents) are formulated for ophthalmic delivery. These pharmaceutical ophthalmic formulations can be in the form of a liquid, an ointment, a gel, an aerosol, a mist, an emulsion, a suspension, a polymer, a film, a paste, or a solid, and can be applied directly to the ocular surface. For example, the compositions are formulated for topical administration to the eye or region of the eye. For example, the formulation can comprise one or more tear substitutes. The formulation alternatively comprises an ophthalmic lubricant. Alternatively, the composition of the invention can be applied to ocular tissue using a device, a contact lens, or a polymer containing a composition according to the invention.

Optionally, such compositions further contain a compound selected from the group consisting of a physiological acceptable hyaluronic acid, glycerin, cyclodextrin, carbopol-methyl cellulose, carboxymethylcellulose (CMC), salt, or petroleum.

The pH of the formulation is preferably between about 5.5 and 7. Preferably, the formulation is an aqueous formulation. The formulation can be in the form of a single dose unit or in the form of a multi-dose system.

The invention also provides methods for treating dry eye and related conditions of the ocular surface. Such methods involve administering to the eye of a subject in need thereof an ophthalmic formulation comprising an effective amount of one or more immune regulatory agents selected from the group consisting of stimulators and activators of T_{reg}, B_{reg}, or type 2 immunity associated immune cells, including ILC2s, Th2, and, alternatively, activation of macrophages (AAMacs, M2). Immune regulatory agents include proteins, peptides, antibodies, antibody fragments, aptamers, nucleic acids, carbohydrates, antisense molecules, RNAi molecules, or any other compounds that partially or fully activate and/or stimulate T_{reg}, B_{reg}, ILC2s, Th2, or AAMacs (M2) cell differentiation, expansion, and function. The immune regulatory agent preferably is administered locally to the ocular surface. Preferably, the amount of the immune regulatory agent employed is effective to stimulate and/or activate T_{reg}, B_{reg}, ILC2s, Th2, or AAMacs (M2) cell differentiation, expansion, and function.

An immune regulatory agent of present invention can be any molecule that partially or fully activates, stimulates, or agonizes biological activity of a molecular component of signaling mediated by the receptor of soluble lymphotoxin alpha (LT-α3), IL-33, IL-7, GM-CSF, IL-3, IL-10, IL-4, IL-13, IL-5, IL-9, or another cytokine or growth factor that is important for the stimulation, activation, differentiation, or immune function of T_{reg}, B_{reg}, or type 2 immunity. The immune regulatory agent, e.g., a recombinant human IL-33, binds to the receptor (ST2 receptor) on a cell expressing ST2 in an ocular tissue, such as ILC2s and Th2, and stimulates ILC2 population expansion and production of effector cytokines, for example IL-4, IL-5, IL-13, and/or IL-9. These type 2 associated effector cytokines further stimulate AAMacs induction and activation, resolution of inflammation, promotion of tissue repair and wound healing, enhancement of goblet cell differentiation, and mucin production, resulting in restoration of tissue homeostasis ocular surface health. Alternatively, an immune regulatory agent of the invention can be any substance that partially or fully activates, stimulates, or agonizes biological activity of IL-33, IL-7, GM-CSF, IL-3, LT-α3, IL-10, IL-4, IL-13, IL-5, IL-9, or other cytokines associated with activation of type 2 immunity and/or T_{reg} or B_{reg} cells. An ophthalmic formulation of the immune regulatory agent, e.g., an ophthalmic formulation of a recombinant human IL-7, acts on ILC2s and IL-22-producing ILCs, which require IL-7 for development and differentiation, the result of which is production of IL-4, IL-13, and IL-22.

In certain preferred embodiments of present invention, the immune regulatory agent is an activator of LT-α3-mediated signal transduction. Preferably, the amount of the immune regulatory agent employed is effective to activate LT-α3 mediated signal transduction. The immune regulatory agent can be administered locally to the ocular surface. In some of these embodiments, the activator of LT-α3-mediated signal transduction is a recombinant human LT-α3 (rhLT-α3), a modified form of recombinant human LT-α3, truncated rhLT-α3, an analog of human LT-α3, a PEGylated form of rhLT-α3, or post-translationally modified form of rhLT-α3. In other embodiments, the stimulator of lymphotoxin-mediated signal transduction is a molecule such as, for example, an antibody, a protein, a peptide, a small molecule, or an aptamer that binds to the receptors for soluble lymphotoxin (tumor necrosis factor receptor 1(TNFR1) and receptor 2 (TNFR2)) and results in activation of signal transduction, or directly induces signal transduction and activation of the receptors for LT-α3.

In other preferred embodiments of the invention, the immune regulatory agent is an activator of IL-33-mediated signal transduction. The immune regulatory agent can be administered locally to the ocular surface. Preferably, the amount of the immune regulatory agent employed is effective to activate IL-33-mediated signal transduction. In some embodiments, the activator of IL-33 mediated signal transduction is a recombinant human IL-33, a modified form of recombinant human IL-33, truncated recombinant human IL-33, an analog of the human IL-33, a PEGylated form of recombinant human IL-33, or post-translationally modified form of recombinant human IL-33. Alternatively, the activator of IL-33 mediated signal transduction is a molecule such as, for example, a protein, a peptide, a small molecule, or an aptamer that binds to the receptor for IL-33 (a heterodimeric receptor consisting of interleukin-1 receptor-related protein ST2 and the IL-1 receptor accessory protein IL1RacP) and results in receptor activation, or directly induces signal transduction and activation of the IL-33 receptor.

In other preferred embodiments of the invention, the immune regulatory agent is an activator of IL-7-mediated signal transduction. The immune regulatory agent can be administered locally to the ocular surface. Preferably, the amount of the immune regulatory agent employed is effective to activate IL-7 mediated signal transduction. In some embodiments, the activator of IL-7 mediated signal transduction is a recombinant human IL-7(rhIL-7), a modified form of rhIL-7, truncated rhIL-7, an analog of the human IL-7, a PEGylated form of rhIL-7, or post-translationally modified form of rhIL-7. In another aspect, the stimulator of IL-7 mediated signal transduction is a molecule such as, for example, an antibody, a protein, a peptide, a small molecule or an aptamer that binds to the IL-7 receptor (a heterodimer of CD127 and CD132) and results in activation of signal transduction, or directly induces signal transduction and activation of the IL-7 receptor.

In yet other embodiments of the invention, two or more immune regulatory agents are included in combination in the ophthalmic formulation and administered topically to the ocular surface. Exemplary immune regulatory agents to be adapted for ophthalmic formulation and topical administration to ocular surface include, but are not limited to, recombinant human LT-α3 combined with recombinant human IL-33, recombinant human LT-α3 combined with recombinant human GM-CSF, or recombinant human IL-7 combined with recombinant human IL-10.

In still other embodiments of the invention, two or more immune regulatory agents are delivered in combination but as separate ophthalmic formulations that are administered topically to the ocular surface of a subject in need of such treatment. Exemplary immune regulatory agents to be adapted for such ophthalmic formulations and topical co-administration to ocular surface include, but are not limited to, recombinant human LT-α3 in combination with recombinant human IL-33, recombinant human LT-α3 in combination with recombinant human GM-CSF, or recombinant human IL-7 in combination with recombinant human IL-10. When such combinations are delivered, each immune regulatory agent may be delivered substantially simultaneously to the subject's affected eye. In other embodiments, the immune regulatory agents are delivered at different times and/or using different dosing regimens.

Dry eye is a multifactorial disease. Dry eye and related conditions of the ocular surface to be treated in accordance with the invention include aqueous deficient dry eye, evaporative dry eye, conjunctivitis sicca, Meibomian gland dysfunction (MGD), blepharitis, and contact lens discomfort. In some variations, the dry eye to be treated is attributable to ocular surface inflammation, autoimmune diseases such as Sjogren's syndrome, and Systemic Lupus Erythematosus (SLE). In some variations, the dry eye to be treated is caused by aging, malnutrition, contact lens wearing, excessive computer monitor or terminal use, and/or medication to other diseases or conditions. Optionally, other related ocular surface conditions include, for example, conditions of the ocular surface with reduced levels of LT-α3, GM-CSF, IL-3, IL-10, IL-4, IL-13, IL-5, or IL-9 in the tear film and having symptoms including, but not limited to, dryness, discomfort, burning, itching, irritation, inflammation, skin abnormalities surrounding the eye, photophobia, blurred vision, and contact lens intolerance.

The foregoing and other aspects of the invention will become more apparent from the following detailed description and the claims. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE DRAWINGS

A brief summary of each of the figures and tables described in this specification are provided below. This application contains at least one figure executed in color. Copies of this document with color drawings will be provided upon request and payment of the necessary fee, if required.

FIG. 1 shows scatter plots between IL-10 and IL-3, IL-4, IL-5, Lymphotoxin alpha (LT-alpha) and GM-CSF (CSF2). All biomarker concentrations are in Log 10 (pg/mL) scale. Strong correlations are observed among cytokines associated with regulatory T cells or type 2 immunity, including Lymphotonxin alpha (TNFbeta), IL-10, GM-CSF (CSF2), IL-3, IL-4, and IL-5. Biomarker data shown were control non-dry eye subjects (11 eyes) and dry eye subjects (102 eyes).

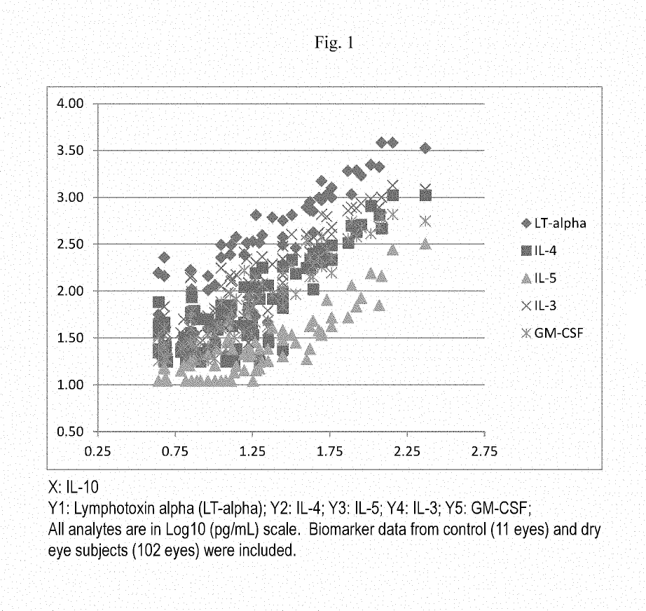

TABLE 1
Significantly lower levels (P <= 0.001) of growth factors and cytokines that are associated with Treg,
Breg and ILC2s, Th2, and AAMacs cells were found in tear fluid collected from dry eye subjects compared with
non-dry eye normal controls, in contrast, significantly higher level of inflammatory cytokine IL-8 was found in dry
eye subjects compared with Non-dry eye normal controls. T test comparison results are listed here with GM-CSF,
IL-3, IL-4, IL-5, IL-10 and Lymphotoxin 3 alpha (TNFbeta). T test results with IL-8 are listed as well. Subjects with
dry eye were recruited with the following criteria: dry eye symptoms for at least 6 months, Ocular Surface Disease Index
(OSDI) total score equal or greater than 23, tear break up time (TBUT) less than 5 seconds, and Schirmer test without
anesthesia for 5 minutes less than 10. Non-dry eye normal control subjects met the criteria of no ocular dry eye symptom
(OSDI less than 13) and no corneal staining and TBUT >= 7. There were 35 DED subjects and 6 control subjects.

| Analyte | t statistics | p value | Mean (DED) | Mean (Control) | unit |
|---|---|---|---|---|---|
| GM-CSF (CSF2) | −3.547 | <0.001 | 49.1 | 186.4 | pg/mL |
| IL-3 | −5.061 | <0.0001 | 63.8 | 342.6 | pg/mL |
| IL-4 | −8.515 | <0.0001 | 18.6 | 226.5 | pg/mL |
| IL-5 | −3.776 | <0.001 | 14.0 | 50.3 | pg/mL |
| IL-10 | −3.521 | <0.001 | 12.4 | 40.5 | pg/mL |
| Lymphotoxin alpha | −3.638 | <0.001 | 93.6 | 865.1 | pg/mL |
| (TNFbeta) | | | | | |
| IL-8 | 2.341 | 0.024 | 698 | 269 | pg/mL |

TABLE 2
Clinical parameters of the DED group and non-DED control subject group.

| | Control | | DED | |
|---|---|---|---|---|
| | Mean | Range | Mean | Range |
| OSDI | 2.4 | 0-8 | 49.5 | 25-89.6 |
| Corneal Staining | 0 | 0 | 5 | 3-10 |
| TBUT | 8.3 | 7.2-11.6 | 3.7 | 2.6-4.9 |
| Schirmer's test | 27 | 8-35 | 6.2 | 1-22 |

### DETAILED DESCRIPTION

### Dry Eye

The term dry eye disease, also called tear-dysfunction, Keratoconjunctivitis sicca (KCS), keratitis sicca, sicca syndrome, xerophthalmia, dry eye syndrome (DES), or simply “dry eyes”, includes a heterogeneous group of ocular surface conditions each associated with a different cause, such as aging, malnutrition, contact lens wearing, excessive use of computer or monitor, systemic autoimmunity in Sjogren's syndrome (SS) leading to ocular dryness, and obstruction of the meibomian gland in meibomian gland dysfunction (MGD) leading to changes in the composition and reduced quality of the tear film and ocular surface damage. All are characterized, however, by the development of ocular symptoms, accompanied by various clinical signs and pathological changes, including reduced tear production, unstable tear layer, irregular corneal surface, loss of goblet cells on the conjunctival epithelia, hyperplasia and sensitization of corneal nerve endings, and development of corneal epitheliopathy. Typical dry eye symptoms are ocular dryness, pain, burning, and sandy gritty sensation and eye irritation that gets worse as the day goes on.

The tear film is constituted by three layers: (1) a lipid layer, produced by the meibomian glands; (2) an aqueous layer, produced by the main and accessory lacrimal glands; and (3) a hydrophilic mucin layer, produced by the conjunctival goblet cells. Any abnormal composition or production of one of these three layers results in an unstable tear film and symptoms of dry eye. According to the report of Dry Eye WorkShop (DEWS), DED is classified into two major types: aqueous-deficient and evaporative dry eye.

In addition to ocular symptoms, moderate to severe dry eye is an ocular surface epithelial disease. The pathogenesis is a multifactorial process, including activating stress pathways in the ocular surface epithelia by the hyperosmolar tear film and cytokines produced by epithelial cells, resident immune cells, and infiltrating CD4 T cells producing IL-17 and interferon (IFN)-γ, leading to ocular surface and lacrimal gland inflammation, reduced tear production, dysregulated epithelial homeostasis, differentiation, and wound healing, enhanced apoptosis, conjunctival goblet cell loss, and reduced mucin production.

Aging is one of the most common causes of dry eyes. About half of all people who wear contact lenses complain of dry eyes. Certain drugs, such as antihistamines, nasal decongestants, beta-blockers, isotretinoin, sedatives, diuretics, tricyclic antidepressants, antihypertensives, and oral contraceptives can cause or worsen DED condition.

Significant progress has been made towards identifying and characterizing the underlying inflammation in the ocular surface in DED through clinical and preclinical research, which has led to better understanding of the key role of inflammation in DED pathogenesis and clinical manifestation. An increasing body of clinical and preclinical evidence implicates specific cytokines and cell subsets as drivers of pathogenesis in DED. It is thought that desiccating stress provokes stress and inflammation at the epithelium of ocular surface, which in turn lead to activation and subsequent expansion of pathogenic IFN-γ-secreting T helper (Th)-1 cells and IL-17-secreting Th17 cells. As an important proinflammatory Th1 cytokine, IFN-γ is known to induce cell surface expression of MHC II, ICAM-1, and a number of co-stimulatory and adhesion molecules in epithelial cells and other cell types, thus enhancing immune activation. IFN-γ has also been shown playing a pivotal role in promoting apoptosis and squamous metaplasia of the ocular surface epithelia in DED. In an experimental dry eye model, IFN-γ induces corneal epithelial apoptosis through activating the apoptotic pathway. Elevated tear level of IFN-γ has also been associated with squamous metaplasia of the ocular surface epithelia in dry eye. In the conjunctival epithelium in a dry eye model, IFN-γ decreased conjunctival goblet cell density and increased expression levels of cornified envelope precursor protein SPRR-2 and other epithelial differentiation-related proteins. It is thought that the Th17 cytokine, IL-17 promotes corneal epithelial barrier disruption. Th-17 cells are IL-17 producing CD4+ T-cell subset distinct from traditional Th-1 and Th-2 lineages, and they have been linked to several autoimmune and autoinflammatory diseases, including DED.

### Goblet Cells and Mucins

Goblet cells (GCs) are simple columnar secretory epithelial cells that secrete gel-forming mucins and are widely distributed throughout mammalian mucosal surfaces, such as gastrointestinal, urogenital, respiratory tracts, and conjunctiva. Mucins produced by goblet cells play critical roles in hydrating, lubricating, and clearing pathogens from the underlying epithelium. Thus, the importance of goblet cells as major producers of mucins is well established. The number and density of functional goblet cells, and the types, amount, and rate of mucins produced are regulated under normal physiological conditions. Alteration of them is associated with pathological conditions, such as hypersecretion of mucin in asthma, and depletion or diminished goblet cell density in intestinal diseases such as inflammatory bowel disease.

On the ocular surface, goblet cells are principal mucin-secreting cells in the conjunctival epithelia, and mucins have integral functions in lubricating the ocular surface epithelia during blinking, stabilizing the tear film, and serve as a physical barrier to external pathogen and particles.

Loss of goblet cells, alteration in goblet cell function, and thus diminished mucin secretion in the ocular surface, are associated with unstable tear film and vulnerable ocular surface. It is well established that goblet cell density and mucin production are reduced in dry eye disease. Loss of goblet cells and depletion of mucin in tear film have been reported moderate to severe dry eye and a number of related conditions of the ocular surface, including Sjögren's syndrome, neutrophilic keratitis, Stevens-Johnson syndrome, ocular mucous membrane pemphigoid, and graft-vs-host disease, where lack of a stable tear film may lead to corneal ulceration and perforation.

IL-13, a type 2 immunity associated cytokine produced by ILC2s and Th2 T cells, can induce goblet cell differentiation and mucus production. On the other hand, IFN-γ, a type 1 immunity-associated cytokine produced by ILC1s and Th1 T cells, can inhibit differentiation of goblet cells and production of mucins.

### Immune Regulation

The ocular surface is continuously exposed to environmental agents such as allergens, pollutants, and microorganisms, which can provoke inflammation. Like other mucosal tissues, such as the intestine, regulation of ocular immune tolerance is important for the homeostasis and protection of the ocular tissues from pathogen invasion and immune-mediated inflammation and immune-mediated injury. There are multiple mechanisms and interactions between different arms and components of the immune system, including the innate immune system, such as innate lymphoid cells (ILCs), tissue-resident mononuclear phagocytes (e.g., macrophages and Dendritic Cells (DCs)), and the adaptive immune system, including T cells and B cells. In the ocular surface, the presence of commensal microbiota and associated mucosal tolerance is also important for the homeostasis of the immune system and tissue. Among T cells, there are naïve T cells, effector T cells (T_{eff}) and regulatory T cells (T_{reg}), and similarly, there are antibody-producing B cells, effector B cells (B_{eff}) and regulatory B cells (B_{reg}).

The dynamic interplay between the regulatory cells and effector cells of the adaptive immune system, such as T_{reg} and T_{eff}, governs the balance between tolerance and effector immune responses. Perturbations of T_{reg} frequency and function or imbalances in T_{reg}/T_{eff} levels are associated with the breakdown of immune tolerance and the development of autoimmunity.

### Regulatory T Cells (T_{reg})

Regulatory T cells (T_{reg}) are suppressive T cells that have an essential role in maintaining the balance between immune activation and tolerance. T_{reg} are vital for keeping the immune system in check, helping to avoid immune-mediated pathology and unrestricted expansion of effector T cell populations, including prevention of autoimmune diseases by maintaining self-tolerance, and in suppression of allergy, asthma, and pathogen-induced immunopathology, among many other functions. Defects in T_{reg} function can be an important factor in the development of autoimmunity or in the failure to control immunopathology; thus, enhancement of T_{reg} function either pharmacologically or by cell-based therapy will be an adjunct to the treatment of autoimmunity. T_{reg} produce IL-10 and it is well known that IL-10 is an important mediator of T_{reg} suppression.

It is recently reported that T_{reg} also play an important role in tissue repair, in additional to immune regulation and suppression of autoimmune and inflammation.

### Th1, Th2, and Th17 Cells

CD4+ T cells are commonly divided into regulatory T cells (T_{reg}) and T helper (Th) effector cells. Conventional effector T helper (Th) cells can be divided into Th1, Th2, and Th17 helper cells, on the bases of their effector function and cytokines they produce. The interferon (IFN)-γ-producing T helper (Th)1 subset of CD4+ cells and the IL-17-producing CD4+ effector cells (Th17) have the capacity to cause inflammation and autoimmune disease, and Th1 and Th17 helper cells are generally considered inflammatory. IFN-γ-producing Th1 cells contribute to the elimination of intracellular pathogens and are involved in cell-mediated and delayed-type hypersensitivity responses. Th17 helper cells mediate host immunity against extracellular bacteria and fungi and their normal role is to provide anti-microbial immunity at epithelial/mucosal barriers. Below is a brief summary of reported Th cell function:

Th1:
Promote adaptive immunity to intracellular pathogens by producing TNF-α and IFN-γ in response to DC-derived IL-12; and
Promote recruitment of inflammatory myeloid cells.

Th2:
Produce IL-4, IL-5, IL-9, and IL-13;
Promote IL-4-mediated Th2 cell type responses, IL-5-dependent recruitment of eosinophils, IL-13 mediated goblet cells differentiation, and mucus production; and
Promote differentiation of alternatively activated macrophages (AAMacs, M2), thus promote repair of mucosal epithelium.

Th17:
Produce IL-17 in response to DC-derived IL-23 and IL-1beta
Promote innate immunity to fungi and extracellular bacteria; and
IL-17 promotes neutrophil recruitment and the production of antimicrobial peptides from intestinal epithelial cells (IECs).

Th2 cells produce type 2 immunity-associated cytokines, including IL-4, IL-5, IL-9, and IL-13, and are involved in allergic responses and the clearance of extracellular pathogens. Functionally, Th2 cytokines have effects on many cell types in the body as receptors for type 2 cytokines are widely expressed on numerous cell types. They are anti-inflammatory, promote resolution of inflammation, epithelial wound healing, production of extracellular matrix components, goblet cells differentiation and mucin production, and tissue repair. IL-4 signals through STAT6 to upregulate GATA3 expression, the master regulator of Th2 cell differentiation. IL-4 also suppresses Th1 and Th17 cell responses and IFN-γ and IL-17 production. IL-4 and IL-13 are the prototypical direct inducers of AAMacs (M2 macrophages), and thus promote repair of mucosal epithelium. IL-13 can increase epithelial cell mucus production, goblet cell proliferation and mucus production, polarize macrophages to an AAMac phenotype, and increase collagen deposition, among other functions. IL-5 is required for recruitment of eosinophils.

### Innate Lymphoid Cells (ILCs)

Innate lymphoid cells (ILCs) are an emerging family of immune cells and have a fundamental role in the immune system by initiating, regulating, and resolving inflammation. ILCs are preferentially enriched at barrier surfaces of the mammalian body, such as the skin, lung, and intestine, as well as in adipose and some mucosal-associated lymphoid tissues. ILCs are important for protection against pathogens and maintenance of organ homeostasis. ILCs rapidly and directly respond to cytokine and microbial signals and are early and potent innate cellular sources of multiple cytokines, both pro-inflammatory and immunoregulatory. ILCs also directly contribute to the resolution of inflammation by repairing damaged tissues, including the lung, various lymphoid tissues, and the gastrointestinal tract. These repair processes are essential to limiting sustained inflammation, preventing re-infection, and restoring tissues to a state of homeostasis. Studies in humans have revealed that ILC responses are substantially altered in several disease states, and in particular recent research has identified a critical role for ILCs in cytokine-mediated regulation of epithelial cell barrier integrity.

The family of ILCs encompasses three subsets, termed group 1, 2, or 3 ILCs, on the basis of common expression of surface markers, transcription factors, and cytokines. ILC subsets produce distinct signature cytokines and immunoregulatory molecules in response to changes in the cytokine microenvironment, as summarized below.

ILC1s:
Express T-bet, a Th1-specific T box transcription factor that controls the expression of the hallmark Th1 cytokine, IFNgamma (IFN-γ);
Promote innate immunity to intracellular pathogens by producing TNF-α and IFN-γ in response to DC-derived IL-12; and
Promote recruitment of inflammatory myeloid cells.

ILC2s:
Highly express GATA3;
Produce IL-4, IL-5, IL-9, IL-13, and amphiregulin in response to epithelial cell-derived and myeloid cell-derived IL-25, IL-33, and TSLP;
Promote IL-4 mediated Th2 cell type responses, IL-5-dependent recruitment of eosinophils, and IL-13-mediated goblet cell differentiation and mucus production; and
Promote differentiation of alternatively activated macrophages (AAMacs, M2), thus promoting repair of mucosal epithelium.

ILC3s:
Express retinoic acid-related orphan receptor positive (RORγt);
Produce IL-17 and IL-22 in response to DC-derived IL-23 and IL-1beta;
Promote innate immunity to fungi and extracellular bacteria; and
IL-17 and IL-22 promote neutrophil recruitment and the production of antimicrobial peptides from intestinal epithelial cells (IECs).

Cytokine-producing ILCs share striking similarities in transcriptional control as the CD4 T helper cell lineages, Th1, Th2, and Th17, respectively. Similar to IFN-γ-producing Th1 cells, intraepithelial ILC1 cells are a unique subset of IL-12- and IL-15-responsive IFN-γ-producing cells. ILC1 cells accumulate in inflamed mucosal tissues. Intraepithelial ILC1 cells respond to danger signals originating from both epithelial cells and myeloid cells, for example, when stimulated by a TLR agonist. It was thought that epithelial cells transmit the dominant danger signals when pathogenic attack is limited to the mucosal surface, whereas monocytes and dendritic cell (DC) stimulation of intraepithelial ILC1 cells might prevail when the epithelial barrier is breached, leading to DC activation in the underlying tissue.

Similar to Th2 cells, ILC2 cells produce IL-4, IL-5, IL-9, and IL-13, which are type2 immunity associated cytokines. IL-4 and IL-13 are the prototypical direct inducers of AAMacs (M2 macrophages). They promote resolution of inflammation, epithelial wound healing, extracellular matrix and mucus production, and tissue repair. ILC2-derived IL-13 can increase epithelial cell mucus production, polarize macrophages to an AAMac phenotype and increase collagen deposition. ILC2 cells can also promote Th2 responses either indirectly by IL-13-elicited migration of activated DCs to the lung draining lymph node and subsequent Th2 cell priming, or directly by major histocompatibility complex class II (MHCII)-dependent interactions with CD4 T cells.

Similar to Th17 cells, ILC3 cells respond to myeloid cell- and DC-derived IL-1β and IL-23 and produce IL-17 and/or IL-22. ILC3 cells can limit chronic inflammation by regulating innate and adaptive immune responses. ILC cells regulate homeostasis of myeloid cells through production of granulocyte macrophage colony-stimulating factor (GM-CSF). In the intestine, this process is regulated by macrophage sensing of intestinal commensal bacteria and production of IL-1beta, which can act on ILC3 cells to promote GM-CSF expression. Production of ILC3-derived LTalpha1bate2 or soluble LTalpha3 can promote IgA production by B cells indirectly by modulating stromal cell or DC responses. Production of ILC3-derived GM-CSF can influence myeloid cell homeostasis to subsequently promote T_{reg} responses and tolerance to food antigens.

ILC3 cells also have an important role in regulating tissue repair in the intestine, as reduced numbers of ILC3 cells in intestinal tissues from IBD patients relative to controls were reported. IL-22 produced by ILC3 cells acts on the intestinal stem cell or progenitor compartments to limit apoptosis and preserve intestinal barrier function. Thus, ILC3 cells are implicated in immune homeostasis and pathogenesis, and also protective immunity at mucosal surfaces.

### Macrophages: Classical Activation of Macrophages (CAMs, M1) and Alternative Activation of Macrophages (AAMacs, M2)

Macrophages arise from hemopoietic progenitors which differentiate into tissue macrophages and closely related myeloid dendritic cells. Tissue resident macrophages are present in organs constitutively, in absence of overt inflammation, and perform trophic as well as homeostatic roles in the removal of apoptotic cells, serving as sentinels of injury and infection. They contribute to tissue remodeling, host defense in innate and adaptive immunity, and many disease processes. At sites of injury and infection, macrophages acquire enhanced cytotoxic, antimicrobial, and inhibitory activities, initiate repair, and resolve inflammation. There are three stages with macrophage differentiation and activation: the first stage is differentiation, which depends on growth factors such as GM-CSF or M-CSF; the second stage is macrophage priming, which can be mediated by IFN-gamma or IL-4 and IL-13; and the third stage is macrophage activation, which is stimulated by local signals delivered by a TLR or analogous receptor.

Based on distinct phenotypes, gene expression profiles, and biological functions, macrophages can be classified into two main types:

Classical Activation of Macrophages (CAMs or M1):
Inflammatory macrophages;
IFN-γ-mediated priming of macrophages;
Regulation of gene expression in CAMs by IFN-γ is dependent on STAT-1;
IFN-γ antagonizes the effect of IL-4 on gene expression;
Inflammatory as characterized with inflammatory cytokine secretion and production of nitric oxide; and
Resulting in an effective pathogen killing mechanism.

Alternative Activation of Macrophages (AAMacs or M2):
Anti-inflammatory, immunosuppression, wound healing, tissue repair, and trophic or regulatory macrophages;
IL-4 and IL-13-mediated priming of macrophages dependent on STAT-6;
IL-4 antagonizes the effect of IFN-γ on gene expression;
Preferentially induces Th2-type responses that are predominated by IL-4, leading to IgG class-switching by B cells;
In addition to the pathogen defense, AAMacs clear apoptotic cells and have a high phagocytosis capacity;
Mitigate inflammatory response, producing large amounts of anti-inflammatory mediators (e.g., IL-10, TGF-beta, IL-1Ra, and lipoxins);
Promote wound healing and tissue repair, including production of extracellular matrix (ECM) components as well as angiogenic and chemotactic factors.

The signature cytokines produced by the AAMacs are IL-10 and IL-1Ra. In the lung, it is thought that AAMacs may provide negative regulatory signals to protect the host from overzealous inflammatory responses to environmental stimuli. Recent studies on this cell population have begun to focus on their potential to mediate wound-healing, angiogenesis, and ECM deposition. The immunoregulatory cytokines TGF-beta and IL-10 play an important role in dampening macrophage activation.

### Type 2 Cytokines, Wound Healing, Goblet Cell, and Mucin in the Ocular Surface:

Type 2 immune responses are defined by the cytokines IL-4, IL-5, IL-9, and IL-13. Type 2 immunity induces a complex immune response characterized by eosinophils, mast cells, basophils, ILC2s, IL-4-and/or IL-13-conditioned macrophages (AAMacs or M2), and Th2 cells. The type 2 immune responses promote tissue repair and are involved in wound repair and the resolution phase of inflammation. Type 2 immunity regulates wound repair and tissue regeneration pathways following infection or injury, suppresses type 1-driven autoimmune diseases, promotes antihelminth immunity, neutralizes toxins, and maintains metabolic homeostasis.

Immune cells of the type 2 response include M2 macrophages, Th2 cells, and ILC2 cells, and they have important roles in suppression of the pro-inflammatory axis. Type 2 immune responses promote effective wound healing by suppressing the pro-inflammatory axis that is mediated by M1 macrophages, ILC1 cells, and Th1 and Th17 cells, which could further exacerbate tissue injury if not quickly controlled.

Epithelial cells can also help to guide type 2 responses by producing the alarmins, thymic stromal lymphopoietin (TSLP), IL-25, and IL-33. TSLP regulates type 2 immunity by suppressing DC-derived IL-12 production, whereas IL-25 and IL-33 primarily target ILC2 cells, which secrete large quantities of IL-4, IL-5, IL-13, and IL-9. Type 2 cytokines in turn target epithelial cells, goblet cells, and macrophages, which together stimulate increasing fluid and mucus production, encapsulation and barrier formation, and epithelial cell turnover. Thus, the type 2 immune response facilitates wound repair.

It has been reported that both conjunctival epithelial cells and corneal epithelial cells in human express functional IL-4 receptor alpha (IL-4Rα) and stimulation with its ligands, IL-4 and IL-13, could induce the expression of various genes, e.g., anti-inflammatory molecules and cellular differentiation and proliferation-related molecules. One of them is hyaluronan synthase 3 (HAS3), which is involved in the synthesis of hyaluronan (hyaluronic acid), a major constituent of the extracellular matrix. Compared to the proteins encoded by other members of the HAS gene family, HAS3 is thought to be more of a regulator of hyaluronan synthesis. HA is present at very high level in the eye, functioning as a tissue lubricant and providing mechanical protection. HA plays a major role in maintaining the health of the cornea (clear front of the eye), the retina, and the vitreous gel that fills most of the eye. It also facilitates wound healing and has been used widely in ophthalmic eye drops for dry eye. It has been shown significantly improved corneal epithelial barrier in dry eye. (Ueta, et al., Br J Ophthalmol. 2010 September; 94(9):1239-43.; Uet,M et al., Jpn J Ophthalmol. 2011 July; 55(4):405-10)

IL-4 and IL-13 have been reported to increase goblet cell number and mucin expression in non-ocular mucosa. The importance of IL-13 in goblet cell hyperplasia is supported by studies showing that direct stimulation of primary lung epithelial cells by IL-13 causes an increase in the population of goblet cells. The effects of IL-13 on goblet cell hyperplasia have been extensively studied in the gastrointestinal and respiratory tracts, and it has also been shown that IL-13 in the conjunctival mucosa regulates goblet cell homeostasis. Furthermore, it has been shown in vitro that IL-13 stimulated proliferation of goblet cell and the release of mucin.

### Effector and Regulatory B Cells

Cytokine production by B cells, one of the antibody-independent functions of B cells, is important for multiple aspects of immunity. B cell-derived cytokines, including lymphotoxin, are essential for the ontogenesis, homeostasis, and activation of secondary lymphoid organs, as well as for the development of tertiary lymphoid tissues at ectopic sites.

Based on cytokine producing profile, B cells can be functionally sub-divided into effector B cells and regulatory B cells. Both effector and regulatory B cells can present antigen to CD4+ T cells and can provide co-stimulation and cytokines; however, they play opposing roles in immune responses.

Effector B cells can secrete cytokines such as IFNγ, IL-12, IL-4, and IL-2 that reinforce and stabilize the cytokine profile of effector Th1 and Th2 cells. In addition, the effector B cells can recruit additional naïve T cells into the inflammatory response, as summarized below.
B effector-1 cells primed by T cells in the presence of Th1-type cytokines secrete IFNγ and IL-12p70 and do not secrete significant amounts of IL-4, IL-13, or IL-2 but secrete IL-10, TNFα, and IL-6.
B effector-2 cells, primed by T cells in the presence of Th2-type cytokines, secrete IL-2, lymphotoxin, IL-4, and IL-13, but very little amount of IFNγ and IL-12. B effector-2 cells can also secrete IL-10, TNFα, and IL-6.

Regulatory B cells are B cells with immunosuppressive or regulatory capacity, and have recently emerged as an important factor for maintaining immune tolerance. Similar to those of T_{reg}, their regulatory functions can be mediated by the release of immunosuppressive cytokines or molecules. Through the production of immunoregulatory cytokines, including IL-10, IL-35 and transforming growth factor beta (TGF-β), regulatory B cells suppress immunopathology by prohibiting the expansion of pathogenic T cells and other pro-inflammatory lymphocytes, promotes T_{reg} development and expansion and alters the activity of antigen-presenting DCs.

It is thought that B-cells expressing IL-10 and LT-α3 have regulatory attributes and potential properties of B_{reg}, favoring homeostasis of immune responses and lymphoid microenvironments. It has been suggested that lymphotoxin-expressing B cells, CXCR5+ DCs and CXCL13, can regulate IL-4-producing Th2 differentiation outside the T cell zone. In prostate cancer, B cells recruited by CXCL13 into prostate tumor promote progression of drug resistant cancer by producing lymphotoxin. The crucial immunosuppressive B cells produce lymphotoxin, IgA, IL-10 and PD-L1, the appearance of which depends on TGFβ receptor signaling.

### IL-10

Interleukin 10 (IL-10) is a crucial immune regulatory cytokine and a powerful negative regulator of the immune-mediated inflammation with a broad range of target cell types, primarily of hematopoietic origin (Jankovic D, Kugler D G, Sher A. IL-10 production by CD4+ effector T cells: a mechanism for self-regulation. Mucosal Immunol. 2010 May; 3(3):239-46). IL-10 can be produced by multiple immune cell types, including AAMacs, regulatory B cells, and regulatory T cells, and plays a particularly prominent role in curtailing immune-mediated-inflammation in the context of infection, allergy, and autoimmunity. IL-10 can suppress pro-inflammatory cytokine production by effector lymphocytes and maintain the differentiation of T_{reg}.

IL-10 production by T_{reg} is essential for keeping immune responses in check and limiting inflammation at environmental interfaces, in particular, in the mucosal tissues, such as the gut and lungs. IL-10 is associated with induction and expansion of T_{reg}. Regulatory T cells that express the transcription factor Foxp3 are pivotal in suppressing autoimmune responses. IL-10 acts on regulatory T cells to maintain expression of the transcription factor Foxp3 and its suppressive function.

IL-10 can be produced by IL-4 or IL-13 primed macrophages, AAMacs, and has an anti-inflammatory property. In the intestine, IL-10 produced by lamina propria macrophages plays a key role in maintaining Foxp3 expression in T_{reg} cells during inflammatory responses.

In homeostatic states under normal condition in mucosa, ILC2, NKT, T_{reg}, and Th2 cells produce IL-10, IL-2, IL-4, IL-3, and other type 2 cytokines; thus, there is a balanced presence of Type 1, Type 2, and Type 17, as evidenced by the balanced ratios of IFNγ/IL-10 and IL-17/IL-10.

IL-10 has been previously associated with non-pathogenic Th17 cells, and suppression of IL-10 production increases those cells pathogenicity. Homeostatic (non-pathogenic) Th17 cells produce IL-10 (IL-17+/IL-10+), while pathogenic Th17 cells do not (IL-17+/IL-10−). Expression of IL-10 was significantly down-regulated in pathogenic Th17 cells. Impaired IL-10 signaling in effector T cells results is augmented in Th17 cell, but not Th1, cell responses, and T_{reg} cells can restrain these Th17 cell responses in an IL-10-dependent manner. Thus, homeostatic Th17 cells have the profile of IL-17+/IL-10+; in contrast, pathogenic Th17 cells have the profile of IL-17+/IL-10−/IFNγ+.

Under physiologic conditions, Th17 cells are found predominantly in the small and large intestine and associated lymphoid tissues where they facilitate production of antimicrobial peptides, enforce integrity of the epithelial barrier, and recruit and activate granulocytes and macrophages to restrain pathogenic bacteria. Anti-inflammatory IL-10 endows T_{reg} with the ability to suppress pathogenic Th17 cell responses. Thus, T_{reg} limit Th17 cell inflammation by serving as principal amplifiers of negative regulatory circuits operating in immune effector cells and IL-10 is one of the critical molecules involved in this regulation.

### IL-33

Interleukin 33 (IL-33) is a recently identified IL-1 family member that signals via a heterodimeric receptor consisting of interleukin-1 receptor-related protein ST2 and the IL-1 receptor accessory protein IL1RacP. IL-33 is expressed by stromal and immune cells, or released by epithelial cells upon tissue damage. IL-33 can act on ILC2 cells directly and induce production of IL-4, IL-5, IL-9, and IL-13. Recent studies have revealed ST2 expression on subsets of regulatory T cells, and for a role for IL-33 in tissue homeostasis and repair that suggests previously unrecognized interactions within these cellular networks. Thus, IL-33 has recently gained interest as a regulator of T_{reg} biology because of its newly discovered function in promoting T_{reg} expansion and function.

### GM-CSF (CSF2)

Granulocyte macrophage colony stimulating factor (GM-CSF, CSF2) is a hematopoietic growth factor that stimulates proliferation and differentiation of hematopoietic progenitor cells. In humans, GM-CSF is localized to a cluster of related genes at chromosome region 5q31, including IL-3, and type 2 cytokines (e.g., IL-4, IL-5, IL-9, and IL-13). The human genes for GM-CSF and IL-3 are closely linked in tandem on chromosome 5, and the high-affinity receptors for human GM-CSF, IL-3 and IL-5 share a common β-subunit.

Cytokines required for monocytic differentiation into DCs, including GM-CSF and IL-4, can be produced by various cell populations, such as Th2 cells, CD3CD56 bright natural killer cells, mast cells, and keratinocytes. Deficiency in GM-CSF production in mice led to decreased T_{reg} numbers and impaired oral tolerance. Ablation of GM-SCF reduced the cell numbers of macrophages and DCs and impaired production of IL-10 and other regulatory factors, leading to disrupted T_{reg} homeostasis in the intestine and thus inflammation in the intestine. It has also been reported that upon exposure to apoptotic cells, GM-CSF-deficient-DCs and macrophages produce an altered cytokine profile that results in decreased T_{reg} and increased Th1 cells.

It was reported that reprogramming of monocytes by GM-CSF contributes to regulatory immune functions during intestinal inflammation and exerts beneficial effects in intestinal inflammation. GM-CSF-activated monocytes accelerate epithelial healing which are accompanied by increased production of IL-10, IL-4, and IL-13, and decreased production of IFN-γ in lamina propria mononuclear cells in vivo. Confirming this finding, GM-CSF-activated monocytes attract T cells and shape their differentiation toward Th2 by upregulating IL-4, IL-10, and IL-13 in T cells in vitro. (Däbritz, et al., J Immunol. 2015, vol. 194(5))

GM-CSF receptor signaling plays a critical role in the molecular pathways that regulate macrophage wound repair activities and promote epithelial wound repair. In a model of vocal fold (VF) injury, treatment with GM-CSF facilitates epithelial wound healing, and promotes regeneration through induction of hyaluronic acids production and gene expressions of extracellular matrix (ECM) components and ECM production-related growth factors, including hyaluronic acid synthase 2 (HAS-2), tropoelastin, MMP-1, HGF, and c-Met. (Lim, et al., PLoS One. 2013; vol. 8(1):e54256).

### Lymphotoxin Alpha (TNF-Beta)

Lymphotoxin-alpha (LT-α) and Lymphotoxin-beta (LT-β) are members of the TNF superfamily designated TNFSF1 and TNFSF3, respectively. LT-α is a soluble protein while LT-β is a type II transmembrane protein. Secreted LT-α assembles as a homotrimer, LT-α3, or as a complex with membrane-associated LT-β to generate two types of heterotrimers, LT-alpha 1/β2 and LT-alpha 2/β1. Lymphotoxins are expressed by RORγt+ ILCs, activated CD4+ T cells, unpolarized IL-2-secreting effector T cells, and Th1 effector cells. Activated naive CD4+ T cells express very high levels of soluble LT-α3. Besides LT-α3 and IL-2, these cells also produce high levels of TNF-alpha together with significant amounts of IFN-γ and IL-13.

In the intestine, soluble LT-α3 produced by RORγt(+) ILC cells controls T cell-dependent IgA induction in the lamina propria via regulation of T cell homing to the gut, thus, soluble LT-α3 is involved in regulating CD4+ T cell, such as T_{reg}, homing and homeostasis. Membrane-bound lymphotoxin beta (LTα1β2) produced by RORγt(+) ILCs is critical for T cell-independent IgA induction in the lamina propria via control of DC functions. Lymphotoxin beta receptor (LTβR) signaling in intestinal epithelial cells promotes self-repair after mucosal damage and LTβR signaling plays a role in epithelial cells in the regulation of intestinal epithelial cell homeostasis to limit mucosal damage. RORγt(+) ILCs, through production of LT, are critical for protection against intestinal pathogens, for maintenance of the epithelial barrier, and for the prevention of systemic dissemination of commensal microbiota.

### IL-22

It is known that upon chemical-, infection-, or irradiation-induced damage of the intestine, ILC3 cells are activated by DC-derived IL-1β, IL-23, TL1A, and RA. Activation of ILC3 cells induces IL-22 production that directly promotes mucus production and epithelial cell repair, in part by acting directly on intestinal stem cells or progenitors.

### IL-7

The development and homeostasis of adaptive and innate lymphocytes is dependent on the stromal cytokine IL-7 (interleukin 7). IL-7 is a hematopoietic growth factor secreted by stromal cells in the bone marrow and thymus. It stimulates the proliferation of lymphoid progenitors. IL-7 is a cytokine important for B and T cell development. ILC cells also express IL-7R and are dependent on IL-7 for their generation. In particular, IL-7 is critical for the differentiation and development of GATA3+ ILC2s and RORγt+ ILC3s. IL-7 stimulates the differentiation of multipotent (pluripotent) hematopoietic stem cells into lymphoid progenitor cells. It also stimulates proliferation of all cells in the lymphoid lineage (B cells, T cells and NK cells). It is important for proliferation during certain stages of B-cell maturation and T and NK cell survival, development, and homeostasis.

IL-7 is also produced by keratinocytes, dendritic cells, hepatocytes, neurons, and epithelial cells but is not produced by normal lymphocytes. IL-7 can be produced locally by intestinal epithelial and epithelial goblet cells, and may serve as a regulatory factor for intestinal mucosal lymphocytes. IL-7 plays an essential role in lymphoid cell survival. IL-7 is necessary in vivo for sustained expression of CD25 at the T_{reg} surface. IL-7 modulation of CD25 expression influenced T_{reg} ability to efficiently bind IL-2 and transduce IL-2 signaling, and drastically affected IL-2 induced T_{reg} expansion in vivo.

### Ocular Immune Tolerance

At mucosal surfaces like the ocular surface, the immune system at physiological state or homeostatic state would not initiate inflammatory immune responses to self-tissues, commensal microbial antigens, or the plethora of antigens constantly present in the environment, but should remain poised to unleash a potent assault on invading pathogens and to respond to environmental insults. Thus, it is necessary for resident immune cells to switch from the homeostatic (often tissue-protective) state to a potent antimicrobial immunity or inflammatory state during local inflammatory process and tissue injury, followed by resolution of acute inflammation, clearance of pathogen and apoptotic cells, wound healing, tissue repair, and restoration of a homeostatic state.

Taken together, it has been proposed that in the mucosa, tissue-resident macrophages and DCs secrete IL-1β, which prompts ILC3 cells to release soluble LT-α3 and GM-CSF. Soluble LT-α3 regulates homing of Tnaïve or T_{reg} cells to the mucosa. T_{reg} cells homing to mucosa or T_{reg} converted from Tnaive are expanded and homeostasis is regulated by GM-CSF, consequently leading to the induction of T_{reg} cells and IL-10 production in the mucosa and maintenance of peripheral immune tolerance and homeostatic state.

### Immune Regulatory Agents

Persons skilled in the art will appreciate from the foregoing that localized stimulation and/or activation of regulatory T cells (T_{reg}) and B cells (B_{reg}), immune cells associated with the type 2 immunity, including ILC2 cells, Th2 cells, and/or AAMacs, can occur at a variety of biological points comprising any one or more of the cells types or interactions described. Stimulation of T_{reg} and/or B_{reg} would result in suppression of inflammation, restoration of peripheral tolerance, and immune homeostasis. Stimulation and/or activation of innate ILC2 cells would induce production of IL-4, IL5, IL-9, and IL-13, thus resulting in Th2 polarization, AAMac induction and activation, resolution of inflammation, promotion of wound healing and tissue repair, and enhancement of goblet cell differentiation and mucin production. Similarly, stimulation and/or induction of AAMacs would lead to clearance of apoptotic cells, suppression of inflammation, production of mucins and other extracellular matrix components, tissue repair, and restoration of tissue homeostasis. For example, stimulation of T_{reg} may occur by induction with topical application IL-10. Or activation of ILC2 cells may occur by stimulating with IL-33 or ST2L receptor signaling, resulting in production of IL-4, IL-5, IL-9, and IL-13. ILC2 cells could also be stimulated with IL-7 or IL-7 receptor signaling transduction. Restoration of peripheral tolerance and immune homeostasis may be achieved by local application of soluble LT-α3, alone or in combination with GM-CSF. Alternatively, ocular surface health can be restored by topical application of soluble LT-α3 in combination with a stimulator of type 2 immunity, for example, an activator of ILC2 and/or Th2 development and production of IL-4, IL-13, IL-5, and IL-9. Stimulation may occur by activating with or through GM-CSF, LT-α3, IL-10, IL-4, IL-13, IL-7, and IL-33 signaling or pathways.

In the context of the invention, an immune regulatory agent is any substance that partially or fully activates, stimulates, or agonizes biological activity of a molecular component of signaling mediated by a receptor of IL-33, IL-7, GM-CSF, LT-α3, IL-10, IL-4, IL-13, or other T_{reg}, B_{reg}, or type 2 immunity stimulating cytokines and growth factors. Alternatively, any immune regulatory agent is any substance that partially or fully activates, stimulates or agonizes biological activity of IL-33, IL-7, GM-CSF, LT-α3, IL-10, IL-4, IL-13, or other type 2 associated cytokines (IL-4, IL-13, IL-5, and IL-9) or regulatory T cells. Thus, activation is the corresponding state stimulated by an activator. A molecular component of signaling mediated by IL-33, IL-7, GM-CSF, LT-α3, IL-10, IL-4, or IL-13 or by agonist of a receptor for IL-33, IL-7, GM-CSF, LTα3, IL-10, IL-4, or IL-13.

It is envisaged that practice of the invention extends to any stimulation or activation of such a receptor known now or in the future.

Suitable classes of activator molecules that stimulate immune regulation, immune regulatory function, or type 2 immunity include recombinant proteins, polypeptides, peptides, peptide mimetic, nucleic acid molecules, and small molecules.

### Ophthalmic Formulations

One or more immune regulatory agents useful in practicing the invention may be formulated in combination with a suitable pharmaceutical carrier. Such formulations preferably comprise therapeutically effective amount of one or more of the immune regulatory agents and an ophthalmically-acceptable carrier (excipient). Such carriers include, but are not limited to, a buffer, osmotic agent, demulcent, surfactant, emollient, tonicity agent, thickening agents and/or preservative component, and combinations thereof. Formulation should suit the mode of administration, and is well within the skill of the art.

For example, the pharmaceutical compositions of the invention can comprise combinations of at least one immune regulatory agent and such carriers, including, but not limited to, saline, buffered saline, dextrose, water, glycerol, and combinations thereof.

In one aspect, the pharmaceutical compositions are formulated for topical administration to the eye. For example, the pharmaceutical compositions are formulated for subconjunctival administration, for example, via eye drops. The pharmaceutical compositions may further comprise a tear substitute. Preferably, the pharmaceutical compositions according to the present invention will be formulated as solutions, suspensions, or other dosage forms for topical administration. Aqueous solutions are generally preferred, based on ease of formulation, as well as a patient's ability to easily administer such compositions by means of instilling one to two drops of the solution in the affected eye(s). However, the compositions may also be suspensions, viscous or semi-viscous gels, or other types of solid or semi-solid compositions. They may also be implanted, for example, as part of a controlled release device or composition.

In general, acceptable ophthalmic carriers may be used, in combination with one or more immune regulatory agents, to adjust the tonicity, pH, stability, viscosity, and sterility of the ophthalmic formulation.

The physiologic pH of blood and tears is approximately 7.4. But the useful range to prevent corneal damage is 6.5 to 8.5. Since the buffer capacity is determined by buffer concentration, the effect of buffers on tonicity must also be taken into account and is another reason that ophthalmic products are usually only lightly buffered. For the adjustment of the pH, preferably to a physiological pH, buffers may especially be useful. The pH of the present solutions should be maintained within the range of 6.0 to 8.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Boric acid is often used to adjust isotonicity in ophthalmic solutions because of its buffering and anti-infective properties. The pH, chemical nature, and volume of the solution to be administered should be considered. Generally, buffers will be used in amounts ranging from about 0.05 to 10 percent by weight. Buffer capacities ranging from 0.01-0.1 are usually adequate for most pharmaceutical solutions.

Any of a variety of carriers may be used in the formulations of the present invention including water, mixtures of water and water-miscible solvents, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, and also other synthetic products, such as polyvinyl alcohol, polyvinylpyrrolidone, polyvinyl methyl ether, polyethylene oxide, preferably cross-linked polyacrylic acid, or mixtures of those polymers. The formulations of the present invention may comprise a compound selected from the group consisting of physiological acceptable salt, poloxamer analogs with carbopol, carbopol/hydroxypropyl methyl cellulose (RP MC), hydroxyethylcellulose, hydroxypropylmethylcellulose, methylcellulose, polyvinyl alcohol, polyvinylpyrrolidone carbopol-methyl cellulose, carboxymethylcellulose (CMC), hyaluronic acid, cyclodextrin, and petroleum.

The concentration of the carrier is, typically, from 1 to 100,000 times the concentration of the active ingredient. Additional ingredients that may be included in the formulation include tonicity enhancers, preservatives, solubilizers, non-toxic excipients, demulcents, sequestering agents, pH adjusting agents, co-solvents, and viscosity building or enhancing agents.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄, or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids, which is equivalent to a 0.9%±0.1% solution of sodium chloride or a 2.5%±0.3% solution of glycerol. An osmolality of the formulation about 225 to about 400 mOsm/kg is preferred, more preferably about 280 to about 320 mOsm.

The topical formulations additionally may comprise a preservative. A preservative may typically be selected from a quaternary ammonium compound such as benzalkonium chloride, benzoxonium chloride, or the like. Where appropriate, a sufficient amount of preservative is added to the ophthalmic composition to ensure protection against secondary contaminations during use caused by bacteria and fungi.

Alternatively, the topical formulations of this invention do not include a preservative.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000, and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to, polysaccharides, such as hyaluronic acid and its salts, chondroitin sulfate and its salts, dextrans, various polymers of the cellulose family, vinyl polymers, and acrylic acid polymers.

The at least one immune regulatory agent may be administered by the use of or in the form of hydrogels, drug-eluting contact lenses, and nanosystems (liposomal systems, dendrimers, solid biodegradable nanoparticles, nanogels), and/or irrigating solutions.

Ophthalmic formulations, eye ointments, creams, salves, powders, solutions, and the like, are also contemplated as being within the scope of this invention.

Furthermore, various other delivery systems are known and can be used to administer the at least one immune regulatory agent. The pharmaceutical composition of the present invention can be administered by any suitable route, including local injection, subconjunctival injection, or intraocular injection, as well as implantation of a sustained release drug device.

### Eye Drops

The use of immune regulatory agent in the eye drop mode for treatment of dry eye condition will enhance their effect by alleviating the bioavailability issue seen in systemic administration.

The eye drop may be formulated with or without one or more tear substitutes. Also provided herein are pharmaceutical or veterinary compositions comprising an effective amount of one or more immune regulatory agents and a tear substitute in a pharmaceutically or veterinarily acceptable carrier for the treatment of dry eye condition. The immune regulatory agent and tear substitute may act synergistically to provide a longer dwell time of the immune regulatory agent on the ocular surface, thus increasing duration and efficacy of action.

A variety of tear substitutes are known in the art and include, but are not limited to: monomeric polyols, such as, glycerol, propylene glycol, and ethylene glycol; polymeric polyols; cellulose esters; dextrans; water soluble proteins; vinyl polymers; and carbomers. Many such tear substitutes are commercially available, which include, but are not limited to, cellulose esters such as Visine Tears®. Tear substitutes may also be comprised of paraffins, such as the commercially available Lacri-Lube® ointments. Other commercially available ointments that are used as tear substitutes such as Refresh PM®.

In one aspect, the tear substitute contains hydroxypropylmethylcellulose. One such tear substitute is Genteal® lubricating eye drops. GenTeal® (CibaVision-Novartis) is a sterile lubricant eye drop containing hydroxypropyl methylcellulose 3 mg/g and preserved with sodium perborate.

The pharmaceutical or veterinary compositions of the invention may comprise combinations of one or more immune regulatory agent and one or more tear substitutes.

The pharmaceutical or veterinary compositions of the invention may comprise combinations of two immune regulatory agents and a tear substitute. Alternatively, the pharmaceutical compositions of the invention may comprise combinations of three immune regulatory agents and a tear substitute.

The topical formulations of the invention may comprise one or more immune regulatory agents and a combination of at least two tear substitutes.

For therapeutic applications, an immune regulatory agent of the invention is administered to a mammal, preferably a human, in a pharmaceutically or veterinarily acceptable dosage form such as those described herein, including those that may be topically administered to a subject's eye in need of treatment for dry eye disease.

For the prevention or treatment of dry eye disease, the appropriate dosage of a particular immune regulatory agent will depend on, for example, the particular disease to be treated, the severity and course of the disease, whether the agent is administered for preventive or therapeutic purposes, previous therapy, the subject's clinical history and response to the agent (if previously administered to the subject), whether the agent is delivered as a monotherapy or as part of a combination therapy, and the discretion of the attending physician. An immune regulatory agent can be administered to the subject at one time or over a series of treatments.

### Kits

Another aspect of the invention concerns articles of manufacture, i.e., kits, containing materials useful for the treatment of dry eye disease disorders described above is provided. Such kits typically comprise a container containing the composition comprising the immune regulatory agent(s) to be administered to provide treatment and instructions for using the accompanying composition. Suitable containers include eyedroppers, tubes, etc. The containers may be formed from any suitable material(s). In any case, the container holds a composition of the invention. The instructions on or associated with the container indicate that the composition is used for treating, for example, dry eye disease. Such instructions often will be in the form of a package insert containing such information as may be required by regulatory authorities

### EXAMPLES

The following Examples are provided to illustrate certain aspects of the present invention and to aid those of skill in the art in its practice. These Examples are in no way to be considered to limit the scope of the invention in any manner, and those having ordinary or greater skill in the applicable arts will readily appreciate that the specification thoroughly describes the invention and can be readily applied to carry out the objects and obtain the ends and advantages mentioned, as well as those inherent therein.

### Example 1

### 1. Study Objectives

To evaluate that topical administration of ophthalmic recombinant human IL-33 (rhIL-33) improves symptoms and signs in dry eye patients.
To identify an appropriate dose for topical administration of ophthalmic rhIL-33 in dry eye patients.

### 2. Background and Rationale

IL-33, through binding to IL-33 receptor on ILC2 cells, stimulates and induces production of type 2 cytokines, including IL-4, IL-5, IL-9, and IL-13, thus promoting tissue repair, wound healing, and resolving inflammation. IL-33 has also been shown to promote T_{reg} expansion and function.

### 3. Study Design

This is a Phase 2, prospective, randomized, double-masked, placebo-control, parallel group, multicenter study in patients with DED. Patients are randomized equally to the following six treatment arms (70 patients per arm) of ophthalmic formulation of recombinant human IL-33 of 1.0 mcg/mL, 4 mcg/mL, 12 mcg/mL, or 25 mcg/mL, twice daily (BID), and placebo control (BID). This study is conducted in compliance with the ethical principles originating in or derived from the Declaration of Helsinki and in compliance with all International Conference on Harmonisation Good Clinical Practice Guidelines.

### 4. Subjects

Eligible dry eye patients are at least 18 years of age with a diagnosis of dry eye for at least 6 months or longer. The major inclusion criteria are:
1) Willingness and ability to read, sign, and date the informed consent and HIPAA documents.
2) Willingness and ability to comply with all study procedures.
3) Normal eye lid anatomy.
4) A history of DED for at least 6 months, supported by a previous clinical diagnosis.
5) Ongoing dry eye, as characterized by:

a) Subject grading total score of >=33 on the OSDI (Ocular Surface Disease Index);

b) Sum of corneal fluorescein staining score of >=6 (National Eye Institute scale); and

c) Tear Breakup Time<5 second.
The major exclusion criteria are:
1) Any ocular condition that, in the opinion of the Investigator, could affect study parameters including, but not limited to, active ocular infection, ocular inflammation, glaucoma, and/or diabetic retinopathy.
2) Any history of an immunodeficiency disorder, positive status for HIV, hepatitis B, C, or evidence of acute active hepatitis A (anti-HAV IgM), or organ or bone marrow transplant.
3) Current or within five years cancer patient in the eye, other organs or blood.
4) Ocular allergy or fibrosis.
5) Currently on or have taken any of the following medication within 30 days of visit 1: topical cyclosporine (Restasis®), ocular corticosteroids, autologous serum, topical ocular antibiotics, topical ocular antihistamines or mast cell stabilizers, or topical or nasal vasoconstrictors.

### 5. Endpoints

Primary endpoints: change in corneal fluorescein staining and dry eye symptom score (ocular pain, or sandy/gritty feeling) from baseline at week 12.

Secondary endpoints: change in other dry eye clinical signs including TBUT, Schirmer's test and conjunctiva staining and in dry eye biomarker test from baseline at week 12.

### 6. Assessments

Safety assessments will include resting blood pressure and pulse rate, clinical laboratory tests, visual acuity, biomicroscopy, conjunctival hyperemia assessment, IOP, and fundus examination.

Efficacy assessment will include both subjective and objective tests. Objective efficacy assessments will include TBUT, corneal staining grading, conjunctival staining grading, and Schirmer's test. Subjective efficacy assessments will include the OSDI questionnaires.

### 7. Statistical Methods

Sample size calculation: accepting 10% margin of error (Type 1, one-sided) to reach 90% confidence level to detect the difference of response between placebo and active group; sample size per treatment arm is set at 70 subjects.

Statistical analysis: the treatment-group comparisons will be performed using analysis of covariance (ANCOVA) for continuous endpoints. For the primary endpoints, the mean change from baseline of corneal fluorescein staining score and dry eye symptom score, ANCOVA model will be used to compare among group differences for the change from baseline to week 12 with covariates including baseline values, treatment group, and stratification factors. Least square (LS) mean difference and its corresponding 90% confidence interval (CI) and p values will be calculated for each treatment comparison.

### Example 2

### 1. Study Objectives

To evaluate that topical administration of ophthalmic recombinant human LT-α3 (rhLT-α3) improves symptoms and signs in dry eye patients.
To identify an appropriate dose for topical administration of ophthalmic rhLT-α3in dry eye patients.

### 2. Background and Rationale

It is thought that soluble LT-α3 produced by RORγt(+) ILC cells is involved in regulating CD4+ T cell, in particular T_{reg} and naïve T cells, homing and homeostasis in the mucosa. RORγt(+) ILC cells, through production of LT-α3, are critical for protection against intestinal pathogens, for maintenance of the epithelial barrier, and for the prevention of systemic dissemination of commensal microbiota.

### 3. Study Design

This will be a Phase 2, prospective, randomized, double-masked, placebo-control, parallel group, multicenter study in patients with DED. Patients will be randomized equally to the following six treatment arms (70 patients per arm) of ophthalmic formulation of LT-α3 of 1.0mcg/mL, 5mcg/mL, 15mcg/mL, or 45mcg/mL, twice daily (BID), and placebo control (BID). This study will be conducted in compliance with the ethical principles originating in or derived from the Declaration of Helsinki and in compliance with all International Conference on Harmonisation Good Clinical Practice Guidelines.

### 4. Subjects

Eligible dry eye patients are at least 18 years of age with a diagnosis of dry eye for at least 6 months or longer. The major inclusion criteria are:

1) Willingness and ability to read, sign, and date the informed consent and HIPAA documents.

2) Willingness and ability to comply with all study procedures.

3) Normal eye lid anatomy.

4) History of dry eye for at least 6 months, supported by a previous clinical diagnosis.

5) Have ongoing dry eye, as characterized by:
a) Subject grading total score of >=33 on the OSDI (Ocular Surface Disease Index);
b) Sum of corneal fluorescein staining score of >=6 (National Eye Institute scale); and
c) Tear Breakup Time<5 second.

The major exclusion criteria are:

1) Any ocular condition that, in the opinion of the Investigator, could affect study parameters, including, but not limited to, active ocular infection, ocular inflammation, glaucoma, and/or diabetic retinopathy.

2) Any history of an immunodeficiency disorder, positive status for HIV, hepatitis B, C, or evidence of acute active hepatitis A (anti-HAV IgM), or organ or bone marrow transplant.

3) Current or within 5 years cancer patient in the eye, other organs or blood.

### 5. Endpoints

Primary endpoints: change in corneal fluorescein staining and dry eye symptom score (e.g., ocular pain or sandy/gritty feeling) from baseline at week 12.

Secondary endpoints: change in other dry eye clinical signs including TBUT, Schirmer's test, and conjunctiva staining and in dry eye biomarker test from baseline at week 12.

### 6. Assessments

Safety assessments will include resting blood pressure & pulse rate, clinical laboratory tests, visual acuity, biomicroscopy, conjunctival hyperemia assessment, IOP and fundus examination.

Efficacy assessment will include both subjective and objective tests. Objective efficacy assessments will include TBUT, corneal staining grading, conjunctival staining grading, snd Schirmer's test. Subjective efficacy assessments will include the OSDI questionnaires.

### 7. Statistical Methods

Sample size calculation: accepting 10% margin of error (Type 1, one-sided), to reach 90% confidence level to detect the difference of response between placebo and active group, sample size per treatment arm is set at 70 subjects.

Statistical analysis: The treatment-group comparisons will be performed using analysis of covariance (ANCOVA) for continuous endpoints. For the primary endpoints, the mean change from baseline of corneal fluorescein staining score and dry eye symptom score, ANCOVA model will be used to compare among group differences for the change from baseline to week 12 with covariates including baseline values, treatment group, and stratification factors. Least square (LS) mean difference and its corresponding 90% confidence interval (CI) and p values will be calculated for each treatment comparison.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A topical ophthalmic formulation for treating dry eye in a mammal, comprising: (a) a therapeutically effective amount of one or more immune regulatory agents selected from the group consisting of stimulators and activators of regulatory T cells (T_{reg}), regulatory B cells (B_{reg}), or type 2 immunity associated immune cells, including group 2 innate lymphoid cells (ILC2s), type 2 CD4+ T helper cells (Th2 cells), and alternative activation of macrophages (AAMacs, M2); and (b) a pharmaceutically or veterinarily acceptable ocular carrier.

2. A formulation according to claim 1 comprising therapeutically effective amount of one or more immune regulatory agents selected from the group consisting of stimulators and activators of T_{reg}, B_{reg}, ILC2 cells, Th2 cells, or AAMacs (M2) cells.

3. A formulation according to claim 1 wherein the each immune regulatory agent is independently selected from the group consisting of a protein, peptide, antibody, antibody fragment, aptamer, nucleic acid, carbohydrate, small molecule, and any other substance that partially or fully activates and/or stimulates T_{reg}, B_{reg}, ILC2 cells, Th2 or AAMacs (M2) cell activity, function, or proliferation.

4. A formulation according to claim 1 wherein each immune regulatory agent is a molecule that partially or fully activates, stimulates, or agonizes biological activity of a molecular component of signaling mediated by a receptor of LT-α3, IL-33, IL-7, GM-CSF, IL-3, IL-10, IL-4, IL-13, IL-5, IL-9, or another cytokine or growth factor that stimulates and/or activates an immune function of T_{reg}, B_{reg}, ILC2 cells, Th2, or AAMacs (M2) cells.

5. A formulation according to claim 4 wherein an immune regulatory agent is a recombinant human IL-33, a modified form of recombinant human IL-33, truncated recombinant human IL-33, an analog of the human IL-33, a PEGylated form of recombinant human IL-33, or post-translationally modified form of recombinant human IL-33.

6. A formulation according to claim 4 wherein an immune regulatory agent is a recombinant human LT-α3 (rhLT-α3), a modified form of recombinant human LT-α3, truncated rhLT-α3, an analog of the human LT-α3, a PEGylated form of rhLT-α3, or post-translationally modified form of rhLT-α3.

7. A formulation according to claim 4 wherein an immune regulatory agent is a recombinant human a recombinant human IL-7(rhIL-7), a modified form of rhIL-7, truncated rhIL-7, an analog of the human IL-7, a PEGylated form of rhIL-7, or post-translationally modified form of rhIL-7.

8. A formulation according to claim 1 wherein the formulation is an aqueous, non-aqueous, gel, ointment formulation, or in the form of a solid, a paste, a liquid, an aerosol, a mist, a polymer, a film, an emulsion, or a suspension.

9. A formulation according to claim 1 further comprises a compound selected from the group consisting of physiological acceptable salt, poloxamer analogs with carbopol, carbopol/hydroxypropyl methyl cellulose (RP MC), hydroxyethylcellulose, hydroxypropylmethylcellulose, methylcellulose, polyvinyl alcohol, polyvinylpyrrolidone carbopol-methyl cellulose, carboxymethylcellulose (CMC), hyaluronic acid, cyclodextrin, and petroleum.

10. A formulation according to claim 1 further comprising an ophthalmically-acceptable excipient.

11. A formulation according to claim 10 wherein the excipient is a buffer, osmotic agent, demulcent, surfactant, emollient, tonicity agent, and/or preservative component.

12. A formulation according to claim 11 wherein the osmolality of the formulation is from about 225 to about 400 mOsm/Kg.

13. A formulation according to claim 11 wherein the osmolality of the formulation is from about 280 to about 320 mOsm/Kg.

14. A formulation according to claim 11 wherein said preservative component is benzalkonium chloride.

15. A formulation according to claim 1, comprising physiologic levels of sodium, potassium, chloride, calcium, magnesium, phosphate, and bicarbonate, and further comprising polysorbate 80, and a borate buffer.

16. An ophthalmic kit comprising the formulation of claim 1 and a means to apply the formulation to the eye.

17. A kit according to claim 16 wherein the application means is an eye dropper, an eye cup, an eye spray, or gel ointment tube.

18. A kit according to claim 16 further comprising a single dose or a multi dose of the formulation in a single container.

19. A method of treating an ocular disease, injury, or disorder comprising administering the formulation of claim 1 to a patient in need of such treatment.

20. A method according to claim 19 wherein the ocular disease is dry eye disease.

21. A method according to claim 19 wherein the ocular disease is one which is associated with Sjogren's syndrome or a systemic autoimmune disease.

22. A method according to claim 19 wherein the ocular disease is one which is due to excessively fast tear evaporation (evaporative dry eyes) or inadequate tear production.

23. A method according to claim 19 wherein the ocular disease, injury, or disorder is one which is attributable to one or more causes selected from the group consisting of aging, contact lens usage, medication usage, and a complication of LASIK refractive surgery.

24. A method according to claim 19 wherein the ocular disease, injury, or disorder is caused by surgery, physical damage to the eye, or by inflammation in the eye.

25. A method according to claim 24 wherein the inflammation in the eye is caused by surgical trauma, dry eye, injury from a chemical, radiation or thermal burn, or penetration of a foreign body.

26. A method of treating an eye wherein its normal immune regulation or wound healing has been disrupted or changed comprising administering to said eye the formulation of claim 1.

27. A method according to claim 26 wherein the eye having disrupted immune regulation or wound healing is due to loss or reduced activity of T_{reg}, B_{reg}, or type 2 immunity in the eye.

28. A method according to claim 26 wherein the eye having disrupted immune regulation or wound healing is diagnosed having lower than normal level(s) of lymphotoxin alpha, IL-10, IL-4, IL-13, IL-5, IL-9, GM-CSF, IL-3 and/or other growth factor or cytokines that are associated with T_{reg}, B_{reg}, or type 2 immunity in the eye.
